(19) United States
(12) Patent Application Publication
(10) Pub. No.: US 2003/0199430 A1
(43) Pub. Date: Oct. 23, 2003

(54) Zinc formulations as a prophylactic treatment for the common cold

(76) Inventors: Richard A. Rosenbloom, Elkins Park, PA (US); Charles A. Phillips, Erwina, PA (US)

Correspondence Address: Kevin J. Dunleavy, KNOBLE & YOSHIDA, LLC., Eight Penn Center, 1628 John F. Kennedy Blvd., Philadelphia, PA, 19103, US

(21) Appl. No.: 10/128,382

(22) Filed: Apr. 23, 2002

(51) Int. Cl. A61K 38/40; A61K 31/555; A61K 31/315; A61K 31/195

(52) U.S. Cl. 514/6; 514/184; 514/561; 514/494

(57) ABSTRACT

Prophylactic treatment to reduce the incidence of common cold using formulations of zinc compounds with selected amino acids is described. The administration of such formulations to healthy individuals resulted in a statistically significant reduction in the incidence of common colds in healthy patients.

### BACKGROUND OF THE INVENTION

1. Field of the Invention

This invention relates to a method of using zinc formulations as a prophylactic treatment. More particularly, this invention relates to methods of using zinc formulations to prevent or reduce the incidence of the common cold.

2. Description of the Prior Art

The management of viral upper respiratory infections, often referred to as “common colds,” has generally been inadequate. The Merck Manual, 16th edition, 1992, pages 190-192 states, “Many viruses cause the common cold, including those in the picomavirus (rhinovirus, certain echoviruses, and coxsackieviruses), influenza, parainfluenza, respiratory syncytial, coronavirus, and adenovirus groups. Most colds (30-50%) are caused by one of the >100 serotypes of the rhinovirus group. Pinpointing the specific cause of each illness by virus isolation or serologic tests is impractical . . . Predisposing factors have not been clearly identified. Chilling the body surface does not by itself induce colds, and an individual's susceptibility is not affected by either health and nutritional status or upper respiratory tract abnormalities (e.g. enlarged tonsils or adenoids). Infection may be facilitated by excessive fatigue, emotional distress, or allergic nasopharyngeal disorders and during the midphase of the menstrual cycle. However, the most important determinant of infection is the presence of specific neutralizing antibody, which indicates previous exposure to a virus and offers relative protection.”

Also according to the Merck Manual, “Many means of preventing acquisition and spread of common colds have been tried, including polyvalent bacterial vaccines, alskalis, citrus fuits, vitamins, ultraviolet lights, and glycol aerosols, but none has been effective. In controlled trials, large (as much as 2 grams per day) prophylactic oral doses of vitamin C have not altered the frequency of acquisition of rhinovirus, common colds or the amount of virus shedding. However, some studies have shown a reduction in duration of disability among persons who took as much as 8 g/day on the first day of disease, although no reduction in virus shedding has been noted.”

The value of nutritional supplements of the element zinc is well established. Several enzymes required for nucleic acid metabolism have been shown to require zinc. In this group are ribonucleic acid (RNA) and deoxyribonucleic acid (DNA) polymerases, deoxythymidine kinase, and reverse transcriptase.

Evidence suggests that nutritional zinc deficiency may be common among the people of many developing countries where they subsist on high cereal protein diets. Marginal zinc deficiency may be widespread even in the United States because of self-imposed dietary restrictions, use of alcohol and cereal proteins, and the use of refined foods, which decrease the intake of trace elements.

The safety of zinc supplements in excess of the amounts found in a normal diet is well documented. Although excessive zinc produces toxic symptoms, such symptoms are rare. Except for extremely large doses, zinc is non-toxic.

G. A. Eby, D. R. Davis, and W. W. Halcomb reported in “Reduction in Duration of Common Colds by Zinc Gluconate Lozenges in a Double Blind Study,” Antimicrobial Agents & Chemotherapy, 1984, 25(1), pp. 20-24; that when modest quantities of zinc are slowly ingested by mouth so that the interior surfaces of the mouth and throat are intermittently bathed in a solution of ionic zinc, both the time course and the severity of the symptoms of the common cold are reduced. This double blind clinical study in sixty-five humans has shown that allowing a tablet containing about 23 mg of elemental zinc, such as zinc gluconate, to slowly dissolve in the mouth once every two hours during 12 to 16 hours a day (the waking hours) reduced the duration of colds from 10.8 days in the untreated group to 3.9 days in the zinc-treated group. In each case, after about one day, the zinc-treated patients had a great reduction in cold symptoms compared to the patients who did not receive zinc.

It has been found that the ingestion of zinc as tablets or capsules which pass directly to the stomach before disintegrating is ineffective for providing a zinc supplement for certain applications, including the control of cold symptoms. In order to solve this problem, U.S. Pat. No. 4,684,528 (J. C. Godfrey) proposes compositions containing a zinc compound such as zinc gluconate, a base material such as a candy or pains for which oral administration of an analgesic is effective; rheumatoid diseases, neuralgia and neuritis; and hyperthermia (common cold, bronchitis, and other central fevers). In this disclosure, the active agent is acetylsalicylate, and the basic amino acid and calcium chloride act to solubilize and stabilize the preparation for formulation in a form suitable for injection.

U.S. Pat. No. 5,422,097 (J. M. Gwaltney, Jr.) discloses a method to treat a patient infected with a common cold, which utilizes both antiviral and anti-inflammatory compounds and employs the simultaneous administration of intranasal and oral medicaments. Also disclosed are investigations in which human volunteers were subjected to a virus challenge with intranasally administered Hank's strain of rhinovirus. Antiviral agents disclosed include interferon a-2 and others. Anti-inflammatory agents disclosed include iprotrophen, naproxen, phenylephrine, chlorpheniramine, and others.

Accordingly, it is an object of certain embodiments of the present invention to provide a method for the prophylactic treatment of the common cold.

It is another object of certain embodiments of the present invention to provide a method for reducing the incidence of common cold by the administration of zinc compounds.

These and other objects of the invention will be apparent from the following general description and examples of the invention.

### SUMMARY OF THE INVENTION

According to the present invention, it has been found that compositions containing a zinc compound and certain amino acids in which the molecular ratio of amino acid to zinc is in the range of two to twenty, can be employed as a prophylactic treatment to reduce the incidence of the common cold. Therefore, in a first aspect, the present invention relates to a method for the prophylactic treatment of a common cold.

### DETAILED DESCRIPTION OF PREFERRED EMBODIMENTS

According to the present invention, it has been found that compositions containing a zinc compound and certain amino acids in which the molecular ratio of amino acid to zinc is in the range of two to twenty, can be employed as a prophylactic acetate, gluconate, ascorbate, citrate, aspartate, picolinate, orotate and transferrin salts, as well as zinc oxide and complexes of divalent zinc with the amino acids. It has been found, however, because of compatibility with the amino acids and the like, that the gluconate, citrate and acetate salts are particularly preferred.

There is nothing about the structures of the effective compounds of this invention or their known nutrient properties that would have enabled one to predict their effectiveness in preventing appearance of cold symptoms in accordance with this invention.

The combination of zinc compound with amino acid may be formulated using an optional base material. The base material which can be utilized as a carrier for the zinc compound and the select amino acid can be a sweetening agent such as a soft or hard candy base; a syrup such as corn syrup; a gum material including chewing gums, or any other form which permits the oral intake of the zinc compound and particularly where the composition is retained in the mouth for a substantial period of time to permit prolonged contact in the mouth with the zinc or provide a slow release of the zinc into the mouth. Preferably the base material is a hard or soft candy base optionally containing a flavoring agent such as a fruit flavor concentrate or a syrup such as a natural or artificially sweetened syrup.

Also in accordance with this invention, a pharmaceutically acceptable carrier can be combined with effective amounts of a combination of the zinc compound, amino acid, and optional base material, according to this invention to provide a palatable oral dosage form for administering to a person to reduce the incidence of a common cold. Accordingly, palatable oral dosage forms according to this invention comprise at least one pharmaceutically acceptable carrier and an effective amount of an effective agent according to this invention. Preferably the effective agent is an aminocarboxylic acid nutrient compound made from a zinc compound and an amino acid as described above. Particularly preferred effective agents are those listed in Table 1. The most preferred effective agent is zinc gluconate-glycine.

One preferred palatable oral dosage form according to this invention is a tablet. A particularly preferred tablet according to this invention comprises a high percentage of at least one effective agent and minor amounts of carrier material acting as binder for the tablet. Suitable binder materials include naturally occurring carbohydrates such as cellulose, starch, galactomannan, fructose, lactose, and sucrose; finely divided ingestible mineral substances such as calcium and magnesium carbonates, calcium and magnesium silicates, calcium and magnesium phosphates, alumina hydrates and hydrotalcite; waxy materials such as beeswax, stearin, stearates of calcium, magnesium, and aluminum, microcrystalline wax and paraffin, and mixtures thereof.

A further palatable oral dosage form according to this invention comprises an effective amount of an effective agent according to this invention in a liquid carrier such as a fruit flavored drink, or in a solid concentrate ready to be converted to such liquid carrier by addition of water at the point of use. Suitable fruit flavored drinks include natural fruit juices such as pineapple juice, apple juice, grape juice, orange juice, grapefruit juice, cranberry juice, and mixtures thereof; reconstituted juices prepared from water and fruit juice concentrates, and fruit juice drinks containing water and at least 10% of natural fruit juice. Preferably the effective agent is an aminocarboxylic acid nutrient compound made from a zinc compound and an amino acid as described above. Particularly preferred effective agents are those listed in Table 1. The most preferred effective agent is zinc gluconate-glycine.

In oral dosage forms according to this invention, the proportions of carrier to effective agent can vary over a broad range in accordance with the kind of carrier selected and the strength desired. Thus the proportion of carrier can be as little as 0.1% by weight, as in a tablet, and as high as 85% or even more, as in a fruit flavored drink.

Also in accordance with this invention, a pharmaceutically acceptable carrier can be combined with effective amounts of an effective agent according to this invention and a flavorant. Accordingly, pleasant tasting oral dosage forms according to this invention comprise at least one pharmaceutically acceptable carrier, an effective amount of an effective agent according to this invention, and a flavorant. Preferably the effective agent is an aminocarboxylic acid nutrient compound made from a zinc compound and an amino acid as described above. Particularly preferred effective agents are those listed in Table 1. The most preferred effective agent is zinc gluconate-glycine.

Preferred flavorants that can be used in a pleasant tasting oral dosage form according to this invention include herbs such as basil, cilantro, dill, oregano, tarragon, and thyme; spices such as cinnamon, clove, ginger, mace, and nutmeg, and essential oils such as oil of lemon, oil of orange, oil of peppermint, and oil of sassafras. In such compositions, the taste characteristics of the first nutrient compound and the second nutrient compound interact in such a way as to produce an overall pleasant tasting composition.

TABLE 1

1. zinc gluconate-glycine
2. zinc acetate-glycine
3. zinc citrate-glycine
4. zinc oxide-glycine
5. zinc acetate-L-leucine
6. zinc gluconate-L-leucine
7. zinc gluconate-D, L-lysine
8. zinc gluconate-L-valine
9. zinc gluconate-D,L-alanine

The following Examples are provided to illustrate the invention without intending to limit its scope, which is defined by the appended claims.

### EXAMPLE 1

A study was conducted to evaluate the benefits of prophylactic administration of Cold-Eeze® to reduce the incidence of the common cold in a school age population of 12-18 year old males and females. Cold-Eeze® is an over-the-counter homeopathic remedy formulated with zinc gluconate-glycine.

Cold-Eeze® lozenges were prepared by titration according to Class F methods of the Homeopathic Pharmacopoeia of the United States. The lozenges were maintained in secure storage at room temperature. One lozenge (13.3 mg of zinc) of Cold-Eeze® per day was administered to approximately 260 subjects of the study during the time period of November to August of the following year.

What is claimed is:

1. A method for the reduction of the incidence of a common cold in a human comprising the step of administering to a human an effective amount of a composition containing a zinc compound and an amino acid to reduce the incidence of a common cold in a human, said amino acid being capable of forming a complex with said zinc compound, and said amino acid being selected from the group consisting of glycine, L-alanine, D,L-alanine, L-2-aminobutyric acid, D,L-2-aminobutyric acid, L-valine, D,L-valine, L-isovaline, D,L-isovaline, L-leucine, D,L-leucine, D-isoleucine, D,L-isoleucine, L-lysine, and D,L-lysine; said composition containing from about 1 mg to about 5 mg of zinc for each gram of said composition, and the molar ratio of said amino acid to zinc being from about 2 to 20.

2. The method of claim 1, wherein an effective amount of said composition provides from about 2 to about 200 mg of zinc per day to said human.

3. The method of claim 1, wherein an effective amount of said composition provides from about 10 to about 150 mg of zinc per day to said human.

4. The method of claim 1, wherein an effective amount of said composition provides from about 20 to about 100 mg of zinc per day to said human.

5. The method of claim 1, wherein said amino acid is glycine.

6. The method of claim 1, wherein said zinc compound is a zinc salt in the form of a sulfate, chloride, acetate, gluconate, ascorbate, citrate, aspartate, picolinate, orotate and transferrin salt.

7. The method of claim 1, wherein said zinc compound is a complex of divalent zinc with said amino acid.

8. The method of claim 1 wherein the zinc compound is zinc gluconate.

9. The method of claim 1 wherein the zinc compound is zinc acetate.

10. The method of claim 1 wherein the zinc compound is citrate.

11. The method of claim 4, wherein said amino acid is glycine.

12. The method of claim 11, wherein the zinc compound is zinc gluconate.
